(19) United States
(12) Patent Application Publication
(10) Pub. No.: US 2026/0232374 A1
(43) Pub. Date: Aug. 13, 2026

(54) CATHETER SYSTEMS FOR FORMING A FISTULA

(71) Applicant: TVA MEDICAL, INC., Franklin Lakes, NJ (US)

(72) Inventors: Cristian Esteban CLAVIJO, Franklin Lakes, NJ (US); Hunter T. KEARNEY, Franklin Lakes, NJ (US); Hoang P. NGUYEN, Franklin Lakes, NJ (US); Alexander PALMER, Franklin Lakes, NJ (US); Mark S. THOMAS, Franklin Lakes, NJ (US)

(21) Appl. No.: 19/160,005

(22) PCT Filed: Mar. 29, 2023
(86) PCT No.: PCT/US2023/016696

§ 371 Date: Aug. 26, 2025

(51) Int. Cl. A61B 18/14; A61B 18/00

(52) U.S. Cl. CPC A61B 18/1492; A61B 2018/00077; A61B 2018/00178; A61B 2018/00404; A61B 2018/00577

(57) ABSTRACT

A catheter including a body, a vessel-fusing element coupled to the body and configured to fuse a pair of body vessels together, and a fistula-forming element positioned within and extending from the vessel-fusing element, the fistula-forming element configured to form a fistula between the pair of body vessels. Additionally two catheters with a vessel-fusing element are claimed, where one catheter also comprises a fistula-forming element.

### TECHNICAL FIELD

The present specification generally relates to catheter systems for forming a fistula and, more specifically, catheter systems that fuse vessels together.

### BACKGROUND

A fistula may be formed between two blood vessels to redirect blood flow from one of the blood vessels to the other. This may be beneficial for medical procedures such as dialysis by increasing blood flow through the dialyzer. When fistulas are formed between the blood vessels, the edges of the fistula may not initially seal, which may lead to some internal bleeding at the fistula site thereby resulting in reduced bloodflow transferred between the two blood vessels.

Accordingly, it may be desirable to provide improved systems for forming a fistula which may reduce fistula site bleeding and improve fistula performance.

### SUMMARY

Embodiments provided herein are directed to addressing the above-noted drawbacks of conventional devices by reducing fistula site bleeding and improving fistula performance.

In one embodiment, catheter includes a body, a vessel-fusing element coupled to the body and configured to fuse a pair of body vessels together, and a fistula-forming element positioned within and extending from the vessel-fusing element, the fistula-forming element configured to form a fistula between the pair of body vessels.

In another embodiment, a catheter system includes a first catheter and a second catheter. The first catheter includes a first body, a first vessel-fusing element coupled to the first body, and a fistula-forming element coupled to and extending from the first vessel-fusing element, the fistula-forming element configured to form a fistula between a pair of body vessels. The second catheter includes a second body, and a second vessel-fusing element coupled to the second body and configured to fuse the pair of body vessels together between the first vessel-fusing element and the second vessel-fusing element.

In yet another embodiment, a method of operating a catheter system, the method includes providing a first catheter, providing a second catheter, and actuating a generator electrically connected to a first electrical pathway to energize the first electrical pathway. The first catheter includes a first body, a first vessel-fusing element coupled to the first body, a fistula-forming element coupled to and extending from the first vessel-fusing element, the fistula-forming element configured to form a fistula between a pair of body vessels, and the first electrical pathway coupled to the first vessel-fusing element. The second catheter includes a second body, and a second vessel-fusing element coupled to the second body and configured to fuse the pair of body vessels together between the first vessel-fusing element and the second vessel-fusing element.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a catheter system for forming a fistula including an ablation catheter and a coaptation catheter, according to one or more embodiments shown and described herein;

FIG. 2 schematically depicts a top view of the ablation catheter of FIG. 1, according to one or more embodiments shown and described herein;

FIG. 3 schematically depicts a cross sectional side view of the ablation catheter of FIG. 2 taken along line 3-3, according to one or more embodiments shown and described herein;

FIG. 4 schematically depicts a top view of the coaptation catheter of FIG. 1, according to one or more embodiments shown and described herein;

FIG. 5 schematically depicts a cross sectional side view of the ablation catheter of FIG. 2 taken along line 5-5, according to one or more embodiments shown and described herein;

FIG. 6 schematically depicts the ablation catheter and the coaptation catheter of the catheter system of FIG. 1 being inserted into adjacent vessels, according to one or more embodiments shown and described herein;

FIG. 7 schematically depicts the ablation catheter and the coaptation catheter of FIG. 6 coapting at a treatment site, according to one or more embodiments shown and described herein;

FIG. 8 schematically depicts a fistula formed at the treatment site of FIG. 7, according to one or more embodiments shown and described herein; and

FIG. 9 schematically depicts another embodiment of an ablation catheter for use with the catheter system of FIG. 1, according to one or more embodiments shown and described herein.

### DETAILED DESCRIPTION

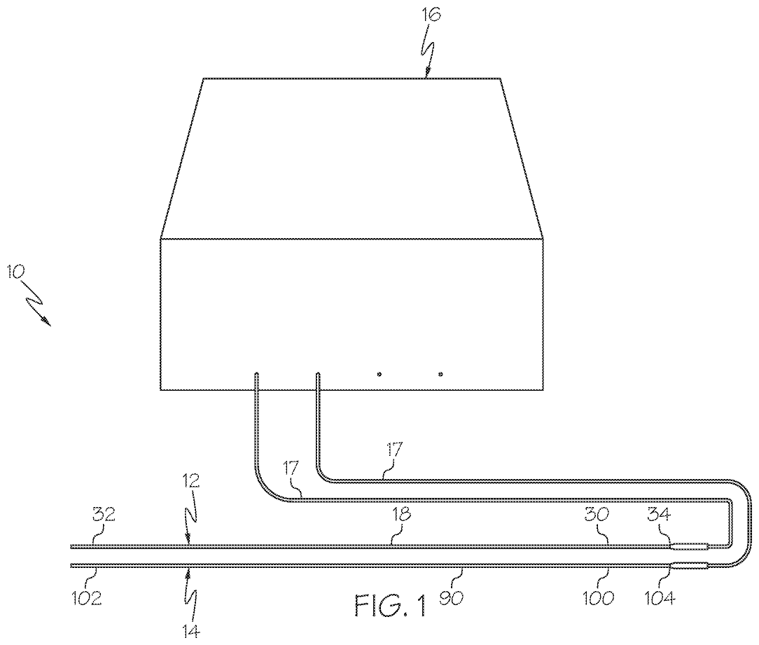

FIG. 1 generally depicts an embodiment of a catheter system for forming a fistula. The catheter system may include an ablation catheter and a coaptation catheter for coapting two body vessels together between the ablation catheter and the coaptation catheter. The ablation catheter may include a body, a vessel-fusing element and a fistula-forming element. The vessel-fusing element is coupled to the body and is configured to fuse a pair of body vessels together. The fistula-forming element is positioned within and extends from the vessel-fusing element and is configured to form a fistula between to the two body vessels. As will be described in greater detail herein, by fusing the vessels together during or before fistula formation, the fused area may reduce or eliminate blood extravasation and provide improved fistula formation and reduced bleeding at the fistula formation site. Various embodiments and benefits will be described in more detail herein.

Referring now to FIG. 1, a catheter system 10 for forming a fistula between a pair of body vessels is depicted. As used herein, “body vessels” may refer to any vessels within a body of a subject, such as blood vessels, though other body vessels are contemplated and possible. For example, the catheter system 10 may be used to form an arteriovenous fistula between an artery and a vein. As will be described in greater detail herein, the catheter system 10 may be used to form the fistula between two body vessels, such as between a first vessel and a second vessel spaced apart from the first vessel, to direct blood flow from one of the first vessel and the second vessel to the other of the first vessel and the second vessel. The catheter system 10 may additionally fuse the first vessel to the second vessel at a fused area around the fistula to prevent the first vessel from separating from the second vessel, thereby preventing internal bleeding through the fistula.

The catheter system 10 may include an ablation catheter 12, a coaptation catheter 14, and a generator 16. For example, the generator 16 may include an electrosurgical generator, such as a direct current electrosurgical generator, waveform generator, or the like. In some embodiments, the generator is a radiofrequency generator. The radiofrequency generator may be a traditional radiofrequency generator, such as the BD ESU-1 Electrosurgical Generator, that generates radiofrequency energy to be transferred to an electrode that is capable of ablating tissue in the body vessel, thereby forming a fistula F. The generator 16 may be provided externally to the ablation catheter 12 and the coaptation catheter 14 and selectively coupled to each of the ablation catheter 12 and the coaptation catheter 14 to provide energy to an electrode disposed therein, as will be discussed in further detail herein. The radiofrequency generator 16 may include a set of cables 17 that transfers the energy from the generator 16 to electrodes included in the ablation catheter 12 and the coaptation catheter 14, as will be described in further detail herein. The cables 17 may selectively attach to the ablation catheter 12 and the coaptation catheter 14 to transfer the energy to the ablation catheter 12 and the coaptation catheter 14. In embodiments, the cables 17 may be fixed to the ablation catheter 12 and the coaptation catheter 14 and selectively attachable to the generator 16, such as via a plug-in connector.

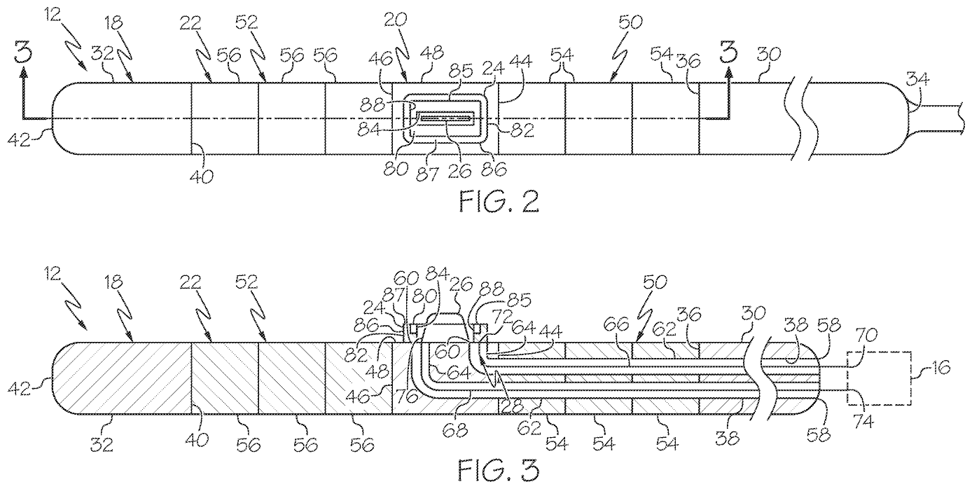

Referring to FIGS. 2-3, the ablation catheter 12 may include an ablation catheter body 18, a conductor housing 20, one or more alignment elements 22, a vessel-fusing element 24, a fistula-forming element 26, and a pair of conductors 28. It is noted that the ablation catheter 12 may include a greater or fewer number of components without departing from the scope of the present disclosure. The ablation catheter body 18 may include a proximal section 30 and a distal section 32 positioned distal to and spaced apart from the proximal section 30. The proximal section 30 may include a proximal end 34 and an opposite distal end 36. The proximal section 30 may at least partially define a pair of conductor lumens 38 extending therethrough from the proximal end 34 to the distal end 36. The distal section 32 may similarly include a proximal end 40 and an opposite distal end 42.

The ablation catheter body 18 may be sized to be advanced through a blood vessel. The distal section 32 and the proximal section 30 may each be shaped and/or sized to aid in advancement of the ablation catheter 12 through a blood vessel. For example, the distal section 32 may be atraumatic for advancement through a blood vessel and may be any suitable shape, such as pointed, tapered, or the like. The ablation catheter body 18 may have any cross-sectional shape and any diameter suitable for intravascular use. The ablation catheter 12 may define additional lumens or other passageways (not shown) extending at least partially along or through the ablation catheter body 18, such as, for example, a guidewire lumen. The ablation catheter body 18 may be formed of any material or combination of materials able to be traversed through a vasculature of a body. For example, the ablation catheter body 18 may include silicone, rubber, or the like.

The one or more alignment elements 22 may extend between the conductor housing 20 and the ablation catheter body 18. In embodiments, the plurality of alignment elements 22 may couple the conductor housing 20 to the ablation catheter body 18. The conductor housing 20 may include a proximal side 44, an opposite distal side 46, and an outer surface 48. The distal side 46 may be positioned distal to the proximal side 44 and the outer surface 48 may extend between the proximal side 44 and the distal side 46. The conductor housing 20 may further define the pair of conductor lumens 38 such that the conductor lumens 38 extend from the proximal end 34 of the proximal section 30 through the conductor housing 20. The conductor housing 20 may be shaped similarly to the ablation catheter body 18 to have a similarly shaped cross-section. For example, the conductor housing 20 may have a circular or oval cross-sectional shape. The conductor housing 20 may be formed of ceramic, polymer, or other type materials. In embodiments, the conductor housing 20 may be electrically and/or thermally insulative.

Still referring to FIGS. 1-3 and as noted above, the ablation catheter 12 may include one or more alignment elements 22 configured to aid in alignment and/or coaptation of the ablation catheter 12 and the coaptation catheter 14. For example, and not as a limitation, the one or more alignment elements 22 may be arranged adjacent to the vessel-fusing element 24, and may include a proximal set of alignment elements 50 positioned between the proximal section 30 of the ablation catheter body 18 and the conductor housing 20, and a distal set of alignment elements 52 positioned between the distal section 32 of the ablation catheter body 18 and the conductor housing 20. The proximal set of alignment elements 50 may be stacked along a length of the ablation catheter 12 so that the proximal set of alignment elements 50 are arranged in a row between the proximal section 30 and the conductor housing 20. The proximal set of alignment elements 50 may be arranged on or within the ablation catheter body 18 proximal to and adjacent the conductor housing 20 and include a plurality of magnetic elements 54 arranged in a longitudinal array along a length of the ablation catheter 12. The distal set of alignment elements 52 may be similarly stacked along a length of the ablation catheter 12 so that the distal set of alignment elements 52 are arranged in a row between the distal section 32 and the conductor housing 20. The distal set of alignment elements 52 may be arranged on or within the ablation catheter body 18 distal to and adjacent the conductor housing 20 and include a plurality of magnetic elements 56 arranged in a longitudinal array along a length of the ablation catheter 12.

The proximal set of alignment elements 50 may further define the pair of conductor lumens 38, as will be described in greater detail herein. The distal set of alignment elements 52 may be stacked along a length of the ablation catheter 12 so that the distal set of alignment elements 52 are arranged in a row between the distal section 32 and the conductor housing 20.

The alignment elements 22 may articulate relative to one another to allow the ablation catheter 12 to bend throughout and maneuver tortuous anatomy. The alignment elements 22 may be sized and shaped, spaced apart, pivotally coupled to one another, or any combination thereof to allow the alignment elements 22 to articulate relative to one another. Each of the proximal set and distal set of alignment elements 50, 52 may have substantially the same dimensions (e.g., height, width, and depth) and substantially the same magnetic strength. In some embodiments, the proximal set and distal set of alignment elements 50, 52 may have a combination of different sized magnets, different shaped magnets, and/or magnets of differing magnetic strength. For example, the individual magnetic elements 56 may have a substantially cubic shape, circular shape, oval shape, or any shape configured to fit within a targeted blood vessel.

The alignment elements 22 elements may have any shape, such square, rectangular, round, oval, etc. In some embodiments, the alignment elements 22 may include a spacer (not shown) coupled between each pair of adjacent alignment elements 22 such that the alignment elements 22 may pivot or move relative to the spacer to allow the ablation catheter body 18 to further articulate.

In some embodiments, the number of the alignment elements 22 of each of the proximal set and distal set of alignment elements 50, 52 may be modified for optimization of magnetic strength for alignment or coaptation purposes. Each of the proximal set of alignment elements 50 and the distal set of alignment elements 52 may comprise any number of individual magnets including one or more magnets, such as 10 or magnets, such as 20 or more magnets, such as 30 or more magnets, etc. In embodiments, the proximal set of alignment elements 50 and the distal set of alignment elements 52 may have different lengths (e.g., different numbers of magnetic elements). For example, the proximal set of alignment elements 50 may have a length that is longer than a length of the distal set of alignment elements 52.

Each of the proximal set of alignment elements 50 and the distal set of alignment elements 52 may be disposed directly adjacent to the conductor housing 20. It is noted that in some embodiments, the proximal set of alignment elements 50 and the distal set of alignment elements 52 may not be positioned directly adjacent the conductor housing 20 but may be spaced from the conductor housing 20.

In embodiments, the alignment elements 22 may be magnets. However, it is contemplated and possible that the alignment elements 22 are formed of any material or include in lieu of or in addition to magnetic elements, one or more mechanisms for coapting with another catheter, such as, for example, balloons, biasing rails, expandable cages, or the like. For further example, the alignment elements 22 may be formed of a ferromagnetic material or an electromagnet configured to be magnetically attracted to another catheter. The other alignment means may be in addition, or alternative, to the proximal set of alignment elements 50 and the distal set of alignment elements 52. The alignment elements 22 may aid in alignment and/or coaptation between the ablation catheter 12 and the coaptation catheter 14.

Still referring to FIGS. 2-3, the pair of conductor lumens 38 may be formed within the proximal section 30, the proximal set of alignment elements 54, and the conductor housing 20. The pair of conductor lumens 28 may be spaced apart from one another (e.g., radially spaced from one another). In the embodiment illustrated, the pair of conductor lumens 38 each include a proximal end 58, a distal end 60, a straight section 62, and an angled or curved section 64. The straight section 62 may extend from the proximal end 34 of the proximal section 30 along a length of the proximal section 30 and through the proximal set of alignment elements 50 into the conductor housing 20. The angled section 64 may extend from the straight section 62 to the distal end 60. The angled section 64 may be entirely defined by the conductor housing 20. However, it is contemplated and possible that the angled section 64 is at least partially defined by the proximal set of alignment elements 22 and/or the proximal section 30 of the ablation catheter body 18. The angled section 64 may extend either obliquely or perpendicularly from the straight section 62 so that the angled section 64 intersects the outer surface 48 of the conductor housing 20 at the distal end 60 of the pair of conductor lumens 38.

The pair of conductors 28 may be coupled between the generator 16 and the vessel-fusing element 24 and fistula-forming element 26 to define electrical pathways that transfer energy from the generator 16 to the vessel-fusing element 24 and the fistula-forming element 26. Accordingly, the pair of conductors 28 may be referred to as electrical pathways 28, where the terms “conductor” and “electrical pathway” may be used interchangeably throughout the ensuing description.

The pair of conductors 28 may be positioned in the pair of conductor lumens 38 extending through the proximal section 30 of the ablation catheter body 18, the proximal set of alignment elements 50, and the conductor housing 20. The pair of conductors 28 may include a first conductor 66, or first electrical pathway 66, operatively coupling the vessel-fusing element 24 to the generator 16 and a second conductor 68, or second electrical pathway 68, operatively coupling the fistula-forming element 26 to the generator 16. The first conductor 66 may be positioned in one of the pair of conductor lumens 38 and the second conductor 68 may be positioned in the other of the pair of conductor lumens 38 so that the first conductor 66 is spaced apart from the second conductor 68.

The first conductor 66 may include a proximal end 70 operatively coupled to the generator 16 and an opposite distal end 72 operatively coupled to the vessel-fusing element 24. The second conductor 68 may include a proximal end 74 operatively coupled to the generator 16 and an opposite distal end 76 operatively coupled to the fistula-forming element 26. FIG. 3 schematically depicts the proximal end 70 of the first conductor 66 and the proximal end 74 of the second conductor 68 coupled to the generator 16. However, the proximal end 70 of the first conductor 66 and the proximal end 74 of the second conductor 68 may be directly connected to the cables 17 of the generator 16 so that the cables 17 transfer energy from the generator 16 to the first conductor 66 and the second conductor 68.

Referring still to FIG. 3, the first conductor 66 and the second conductor 68 are operatively coupled from the generator 16 to the vessel-fusing element 24 and the fistula-forming element 26 so that the pair of conductors 28 are configured to transfer energy from the generator 16 to the vessel-fusing element 24 and the fistula-forming element 26. The conductors 28 may be formed of any material capable of transferring energy (such as radiofrequency energy) from the generator 16, such as copper, aluminum, iron, steel, nickel, zinc, etc. It is noted that though only a single generator is depicted. It is contemplated that each conductor may be coupled to a separate generator.

Referring again to FIGS. 2 and 3, the vessel-fusing element 24 may include a vessel-fusing housing 86 and a conductive plate 85 coupled to the vessel-fusing housing 86. The vessel-fusing housing 86 may include an outer surface 87 and a periphery that surrounds the outer surface 87. The vessel-fusing housing 86 may define a recess 84 configured to receive the fistula-forming element 26, and a plate recess 88 configured to receive the conductive plate 85. Each of the recess 84 and the plate recess 88 may extend through the outer surface 87 and be positioned such that the periphery 82 surrounds the recess 84 and the plate recess 88. In other words, the recess 84 and the plate recess 88 are each spaced apart from the periphery 82 such that the recess 84 and the plate recess 88 does not intersect the periphery 82. The recess 84 may be shaped such that the fistula-forming element 26 can be positioned within the recess 84 without contacting the vessel-fusing element 24. The plate recess 88 may be shaped to receive the conductive plate 85 and be sized so that the plate recess 88 extends around the recess 84. The vessel fusing housing 86 may be made of a number of materials including but not limited to tungsten rhenium, copper, aluminum, iron, steel, nickel, zinc, etc.

The first conductor 66 may be operatively coupled at the distal end 72 to the conductive plate 85, such that the conductive plate 85 is configured to act as a conduit for the energy and transfer the energy to the vessel walls. The conductive plate 85 may have a fusing surface 80. The conductive plate 85 may be positioned within the plate recess 88 of the vessel-fusing housing 86 and coupled thereto by, for example, a press fit. In some embodiments, the conductive plate 85 may be adhered or otherwise affixed within the plate recess 88. The conductive plate 85 may be sized and positioned within the plate recess 88 so that the fusing surface 80 is substantially coplanar with the outer surface 87 of the vessel-fusing housing 86. The fusing surface 80 may be configured to contact the vessel wall to fuse the vessel wall to another vessel wall when energized by the conductors 28, as will be described in further detail herein. For example, the fusing surface 80 may be an exposed ablation surface. The vessel-fusing element 24 may be coupled to and extend from the outer surface 48 of the conductor housing 20 with the fusing surface 80 spaced apart from and facing away from the conductor housing 20. The vessel-fusing housing 86 may be coupled to the conductor housing 20 via any traditional manufacturing technique (e.g., via fasteners, adhesives, of the like). In some embodiments, the vessel-fusing housing 86 and the conductor housing 20 may be integrally formed with one another such as machined from a common block of material, injection molded, or the like.

In embodiments, the vessel-fusing element 24 and the conductive plate 85 may be shaped so that the fusing surface 80 may be flat, curved to be concave or convex, or the like. Referring specifically to FIG. 2, the vessel-fusing element 24 may additionally be shaped so that the vessel-fusing element 24 is rounded at the periphery 82. For example, the vessel-fusing element 24 may have a rounded cross-section, such as a circle or oval, extending from the outer surface 48 of the conductor housing 20. However, it is contemplated and possible that the vessel-fusing element 24 has a shape that is not rounded, such as a square, a rectangle, or the like.

The conductive plate 85 may be formed of metal or any other material capable of transferring energy from the conductors 28 to the vessel wall (e.g., copper, aluminum, iron, steel, nickel, zinc, etc.). The conductive plate 85 may be configured to fuse the walls of two vessels together when the conductive plate 85 is energized by the generator 16 via the first conductor 66. When the generator 16 is activated to send energy to the conductive plate 85 via one of the conductors 28, energy may be supplied to and/or carried from tissue and fluid via the fusing surface 80 to facilitate fusing the body vessels to one another.

Referring still to FIGS. 2-3, the fistula-forming element 26 may be positioned in the recess 84 of the vessel-fusing element 24. The fistula-forming element 26 may be an electrode, such as a leaf spring electrode, such that the fistula-forming element 26 is configured to form a fistula in one or more vessels when the fistula-forming element 26 is energized by the generator 16 via the second conductor 68. The fistula-forming element 26 may extend away radially beyond the fusing surface 80 of the vessel-fusing element 24 so that the fistula-forming element 26 extends away from the conductor housing 20 a distance greater than a distance between fusing surface 80 As depicted in FIG. 3, the fistula-forming element 26 may be arc shaped, though other shapes are contemplated and possible (e.g., rectangular, square, angular, etc.). The size and shape of the fistula-forming element 26 may be varied based on factors including tissue thickness and density, as well as desired fistula size, shape, and location. It is noted that the fistula-forming element is not limited to an electrode as described above, but may include a different cutting/ablation device such as, but not limited to, any electrocautery mechanism, blades, lances, needles, cryogenic-cautery devices, ultrasonic-cautery devices, laser ablation devices, etc. The fistula-forming element 26 may be formed of any conductive material such as, but not limited to, copper, aluminum, iron, steel, nickel, zinc, Nitinol, etc. In embodiments, the fistula-forming element 26 may be resilient or spring to extend radially away from the conductor housing 20.

In embodiments, such as where the fistula-forming element 26 is a leaf spring, the fistula-forming element may be able to bend or deflect to retract into the vessel-fusing housing 86 so as to have a low profile configuration. For example, the fistula-forming element 26 may be held in a low profile orientation via a sleeve (not depicted). During activation of the fistula-forming element 26 to form a fistula, the sleeve may be withdraw, and the bias (such as a natural bias) of the fistula-forming element 26 may cause the fistula-forming element 26 to project out of the vessel-fusing housing 86 when not constrained. Moreover, the bias of the fistula-forming element 26 may aid it in advancing through vessel tissue during ablation.

In some embodiments, the ablation catheter 12 may comprise one or more insulating materials (not shown) which may shield or otherwise protect the ablation catheter 12 and its components from heat generated by the fistula-forming element 26 and the vessel-fusing element 24 during use. The insulating materials may be a coating layer positioned in or on the ablation catheter 12, such as an insulating layer positioned in the recess 84 between the fistula-forming element 26 and the vessel-fusing element 24 to prevent transfer of energy between the fistula-forming element 26 and the vessel-fusing element 24. Insulating materials may additionally or alternatively be positioned in the pair of conductor lumens 38 and/or around the conductors 28 to prevent energy transfer from the energized conductors 28 to the proximal section 30, the proximal set of alignment elements 22, and the conductor housing 20 through which the conductor lumens 38 extend. The insulating materials may be any material capable of thermally insulating the surrounding components such as, for example, ceramic, polymeric materials, etc.

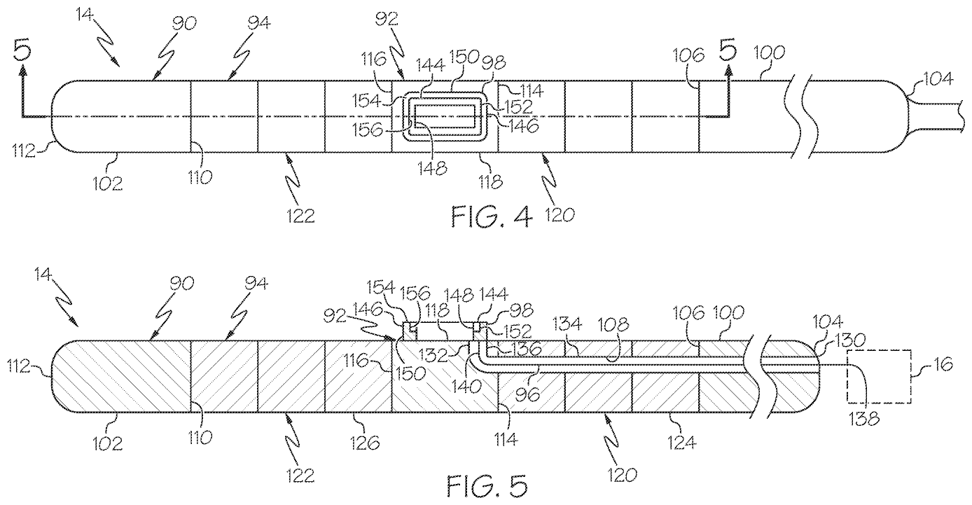

Referring now to FIGS. 4-5, the coaptation catheter 14 may be configured to coapt with the ablation catheter 12 to coapt the two body vessels together between the coaptation catheter 14 and the ablation catheter 12. The coaptation catheter 14 may be substantially similar to the ablation catheter 12 described above. For example, the coaptation catheter 14 may include a coaptation catheter body 90, an conductor housing 92, a plurality of alignment elements 94, an conductor 96, and a vessel-fusing element 98. It is noted that the coaptation catheter 14 may include a greater or fewer number of components without departing from the scope of the present disclosure. The coaptation catheter 14 is substantially similar to the ablation catheter, but as illustrated, may not include a fistula-forming element.

The coaptation catheter body 90 may include a proximal section 100 and a distal section 102 positioned distal to and spaced apart from the proximal section 100. The proximal section 100 may include a proximal end 104 and an opposite distal end 106. The proximal section 100 may at least partially define a conductor lumen 108 extending therethrough from the proximal end 104 to the distal end 106. The distal section 102 may include a proximal end 110 and an opposite distal end 112.

The coaptation catheter body 90 may be sized to be advanced through a blood vessel. The distal section 102 and the proximal section 100 may each be shaped and/or sized to aid in advancement of the coaptation catheter 14 through a blood vessel. For example, the distal section 102 may be atraumatic for advancement through a blood vessel and may be any suitable shape, such as pointed, tapered, or the like. The coaptation catheter body 90 may have any cross-sectional shape and any diameter suitable for intravascular use. The coaptation catheter 14 may define additional lumens or other passageways (not shown) extending at least partially along or through the coaptation catheter body 90, such as, for example, a guidewire lumen. The coaptation catheter body 90 may be formed of any material or combination of materials able to be traversed through a vasculature of a body. For example, the coaptation catheter body 90 may include silicone, rubber, or the like.

The one or more alignment elements 94 may extend between the conductor housing 92 and the coaptation catheter body 90. In embodiments, the plurality of alignment elements 94 may couple the conductor housing 92 to the coaptation catheter body 90. The conductor housing 92 may include a proximal side 114, an opposite distal side 116, and an outer surface 118. The distal side 116 may be positioned distal to the proximal side 114 and the outer surface 118 may extend between the proximal side 114 and the distal side 116. The conductor housing 92 may further define the conductor lumen 108 such that the conductor lumen 108 extends from the proximal end 104 of the proximal section 100 through the conductor housing 92. The conductor housing 92 may be shaped similarly to the coaptation catheter body 90 to have a similarly shaped cross-section. For example, the conductor housing 92 may have a circular or oval cross-sectional shape. The conductor housing 92 may be formed of ceramic, polymer, or other type materials. In embodiments, the conductor housing 92 may be electrically and/or thermally insulative.

Still referring to FIGS. 4 and 5, and as noted above, the coaptation catheter 14 may include one or more alignment elements 94 configured to aid in alignment and/or coaptation of the coaptation catheter 14 and the coaptation catheter 14. For example, and not as a limitation, the one or more alignment elements 94 may include a proximal set of alignment elements 120 positioned between the proximal section 100 of the coaptation catheter body 90 and the conductor housing 92, and a distal set of alignment elements 122 positioned between the distal section 102 of the coaptation catheter body 90 and the conductor housing 92. The proximal set of alignment elements 120 may be stacked along a length of the coaptation catheter 14 so that the proximal set of alignment elements 120 are arranged in a row between the proximal section 100 and the conductor housing 92. The proximal set of alignment elements 120 may be arranged on or within the coaptation catheter body 90 proximal to and adjacent the conductor housing 92 and include a plurality of magnetic elements 124 arranged in a longitudinal array along a length of the coaptation catheter 14. The distal set of alignment elements 122 may be similarly stacked along a length of the coaptation catheter 14 so that the distal set of alignment elements 122 are arranged in a row between the distal section 102 and the conductor housing 92. The distal set of alignment elements 122 may be arranged on or within the coaptation catheter body 90 distal to and adjacent the conductor housing 92 and include a plurality of magnetic elements 126 arranged in a longitudinal array along a length of the coaptation catheter 14.

The proximal set of alignment elements 120 may further define the conductor lumen 108, as will be described in greater detail herein. The distal set of alignment elements 122 may be stacked along a length of the coaptation catheter 14 so that the distal set of alignment elements 122 are arranged in a row between the distal section 102 and the conductor housing 92.

The alignment elements 94 may articulate relative to one another to allow the coaptation catheter 14 to bend throughout and maneuver tortuous anatomy. The alignment elements 94 may be sized and shaped, spaced apart, pivotally coupled to one another, or any combination thereof to allow the alignment elements 94 to articulate relative to one another. Each of the proximal set and distal set of alignment elements 120, 122 may have substantially the same dimensions (e.g., height, width, and depth) and substantially the same magnetic strength. In some embodiments, the proximal set and distal set of alignment elements 120, 122 may have a combination of different sized magnets, different shaped magnets, and/or magnets of differing magnetic strength. For example, the individual magnetic elements may have a substantially cubic shape, circular shape, oval shape, or any shape configured to fit within a targeted blood vessel. There may be any number of individual magnetic elements, such as 10 or magnets, such as 20 or more magnets, such as 30 or more magnets, etc.

The alignment elements 94 may have any shape, such square, rectangular, round, oval, etc. In some embodiments, the alignment elements 94 may include a spacer (not shown) coupled between each pair of adjacent alignment elements 94 such that the alignment elements 94 may pivot or move relative to the spacer to allow the coaptation catheter 14 to further articulate.

In some embodiments, the number of the alignment elements 94 of each of the proximal set and distal set of alignment elements 120, 122 may be modified for optimization of magnetic strength for alignment or coaptation purposes. Each of the proximal set of alignment elements 120 and the distal set of alignment elements 122 may comprise any number of individual magnets including one or more magnets. Moreover, each catheter may comprise any number of individual magnets (e.g., one, two, three, four, five, six, seven, or eight or more, etc.). In embodiments, the proximal set of alignment elements 120 and the distal set of alignment elements 122 may have different lengths (e.g., different numbers of magnetic elements). For example, the proximal set of alignment elements 120 may have a length that is longer than a length of the distal set of alignment elements 122.

Each of the proximal set of alignment elements 120 and the distal set of alignment elements 122 may be disposed directly adjacent to the conductor housing 92. It is noted that in some embodiments, the proximal set of alignment elements 120 and the distal set of alignment elements 122 may not be positioned directly adjacent the conductor housing 92 but may be spaced from the conductor housing 92.

In embodiments, the alignment elements 94 may be magnets. However, it is contemplated and possible that the alignment elements 94 are formed of any material or include in lieu of or in additional to magnetic elements, one or more mechanisms for coapting with another catheter, such as, for example, balloons, biasing rails, expandable cages, or the like. For further example, the alignment elements 94 may be formed of a ferromagnetic material or an electromagnet configured to be magnetically attracted to another catheter. The other alignment means may be in addition, or alternative, to the proximal set of alignment elements 120 and the distal set of alignment elements 122. The alignment elements 94 may aid in alignment and/or coaptation between the ablation catheter 12 and the coaptation catheter 14.

Referring still to FIG. 5, the conductor lumen 108 may include a proximal end 130, a distal end 132, a straight section 134, and an angled or curved section 136. The straight section 134 may extend from the proximal end 104 of the proximal section 100 along a length of the proximal section 100 and through the proximal set of alignment elements 120 into the conductor housing 20. The angled section 136 may extend from the straight section 134 to the distal end 106 of the conductor lumen 108. The angled section 136 may be entirely defined by the conductor housing 92. However, it is contemplated and possible that the angled section 136 is at least partially defined by the proximal set of alignment elements 120 and/or the proximal section 100 of the coaptation catheter body 90. The angled section 136 may extend either obliquely or perpendicularly from the straight section 134 so that the angled section 136 intersects the outer surface 118 of the conductor housing 92 at the distal end 106 of the conductor lumen 108.

The conductor 96 may be coupled between the generator 16 and the vessel-fusing element 98 to define an electrical pathway that transfers energy from the generator 16 to the vessel-fusing element 98. Accordingly, the conductor 96 may be referred to as an electrical pathway, where the terms “conductor” and “electrical pathway” may be used interchangeably throughout the ensuing description.

The conductor 96 may be positioned in the conductor lumen 108 extending through the proximal section 100 of the coaptation catheter body 90, the proximal set of alignment elements 120, and the conductor housing 20. The conductor 96 may be positioned in the conductor lumen 108 and operatively couple the vessel-fusing element 98 to the generator 16. The conductor 96, or electrical pathway 96, may include a proximal end 138 operatively coupled to the generator 16 and an opposite distal end 140 operatively coupled to the vessel-fusing element 98 so that the conductor 96 is configured to transfer energy from the generator 16 to the vessel-fusing element 98. FIG. 5 schematically depicts the proximal end 138 of the conductor 96 to the generator 16. The conductor 96 may be formed of any material capable of transferring energy (such as radiofrequency energy) from the generator 16, such as copper, aluminum, iron, steel, nickel, zinc, etc.

The vessel-fusing element 98 may include a vessel-fusing housing 150 and a conductive plate 152 coupled to the vessel-fusing housing 150. The vessel-fusing housing 150 may include an outer surface 154 and a periphery 146 that surrounds the outer surface 154. The vessel-fusing housing 150 may define an opening 148, such as within the conductive plate 152, configured to receive the fistula-forming element 26 of the ablation catheter 12 when the coaptation catheter 14 coapts with the ablation catheter 12, and a plate recess 156 configured to receive the conductive plate 152. The vessel fusing housing 150 may be made of a number of materials including but not limited to tungsten rhenium, copper, aluminum, iron, steel, nickel, zinc, etc.

Each of the opening 148 and the plate recess 156 may extend through the outer surface 154 of the vessel-fusing housing 150 such that the periphery 146 surrounds the opening 148 and the plate recess 156. In other words, the opening 148 and the plate recess 156 may be spaced apart from the periphery 146 such that the opening 148 and the plate recess 156 do not intersect the periphery 146. The opening 148 may be shaped such that the fistula-forming element 26 of the ablation catheter 12 can be positioned within the opening 148 without contacting the vessel-fusing element 98 of the coaptation catheter 14. The plate recess 156 may be shaped to receive the conductive plate 152 and be sized so that the plate recess 156 extends around the opening 148.

The conductor 98 may be operatively coupled at the distal end 132 to the conductive plate 152 so that the conductive plate 152 is configured to act as a conduit for the energy and transfer the energy to the vessel walls. The conductive plate 152 may include a fusing surface 144. The conductive plate 152 may be positioned within the plate recess 156 of the vessel-fusing housing 150 and coupled thereto by, for example, a press fit, adhesive, o the like. The conductive plate 152 may be sized and positioned within the plate recess 156 so that the fusing surface 144 is substantially coplanar with the outer surface 154 of the vessel-fusing housing 150. The fusing surface 144 may be configured to contact the vessel wall to fuse the vessel wall to another vessel wall when energized by the conductor 96, as will be described in further detail herein. For example, the fusing surface 144 may be an exposed ablation surface. The vessel-fusing element 98 may be coupled to and extend from the outer surface 118 of the conductor housing 92 with the fusing surface 144 spaced apart from and facing away from the conductor housing 92. The vessel-fusing housing 150 may be coupled to the conductor housing 92 via any traditional manufacturing technique (e.g., via fasteners, adhesives, of the like). In some embodiments, the vessel-fusing housing 150 and the conductor housing 92 may be integrally formed with one another such as machined from a common block of material, injection molded, or the like.

The vessel-fusing element 98 and the conductive plate 152 may be shaped to be complementary to the shape of the vessel-fusing element 24 of the ablation catheter 12. For example, the fusing surface 144 may be flat, curved to be concave or convex, or the like similar to the fusing surface 80 of the ablation catheter 12 so that the vessel-fusing element 24 of the ablation catheter 12 may be fully engage with the vessel-fusing element 98 of the coaptation catheter 14 when the coaptation catheter 14 coapts with the ablation catheter 12. For example, when the fusing surface 80 of the vessel-fusing element 24 of the ablation catheter 12 is convex, the fusing surface 144 of the vessel-fusing element 98 of the coaptation catheter 14 is concave such that they surface nest together.

As depicted in FIG. 4, the vessel-fusing element 98 and the conductive plate 152 may be shaped so that the fusing surface 144 may be flat, curved to be concave, convex, or the like. In embodiments, the vessel-fusing element 98 may additionally be shaped so that the vessel-fusing element 98 is rounded at the periphery 146. For example, the vessel-fusing element 98 may have a rounded cross-sectional shape, such as a circle or oval, extending from the outer surface 118 of the conductor housing 92. However, it is contemplated and possible that the vessel-fusing element 98 has a shape that is not rounded, such as a square, a rectangle, or the like.

Referring again to FIGS. 1, 4, and 5, the conductive plate 152 may be formed of metal or any other material capable of transferring energy from the conductor 96 to the vessel wall (e.g., copper, aluminum, iron, steel, nickel, zinc, etc.). The conductive plate 152 may be configured to fuse the walls of two vessels together when the conductive plate 152 is energized by the generator 16 via the conductor 96. When the generator 16 is activated to send energy to the conductive plate 152 via the conductor 96, energy may be supplied to and/or carried from tissue and fluid via the fusing surface 144 to facilitate fusing the body vessels.

In some embodiments, the coaptation catheter 14 may comprise one or more insulating materials (not shown) which may shield or otherwise protect the coaptation catheter 14 and its components from heat generated by the vessel-fusing element 98 during use. The insulating materials may be a coating layer positioned in or on the coaptation catheter 14, such as an insulating layer in the conductor lumen 108 and/or around the conductor 96 to prevent energy transfer from the energized conductor 96 to the proximal section 100, the proximal set of alignment elements 120, and the conductor housing 92 through which the conductor lumen 108 extends. The insulating materials may be any material capable of thermally insulating the surrounding components such as, for example, ceramic, polymeric materials, etc.

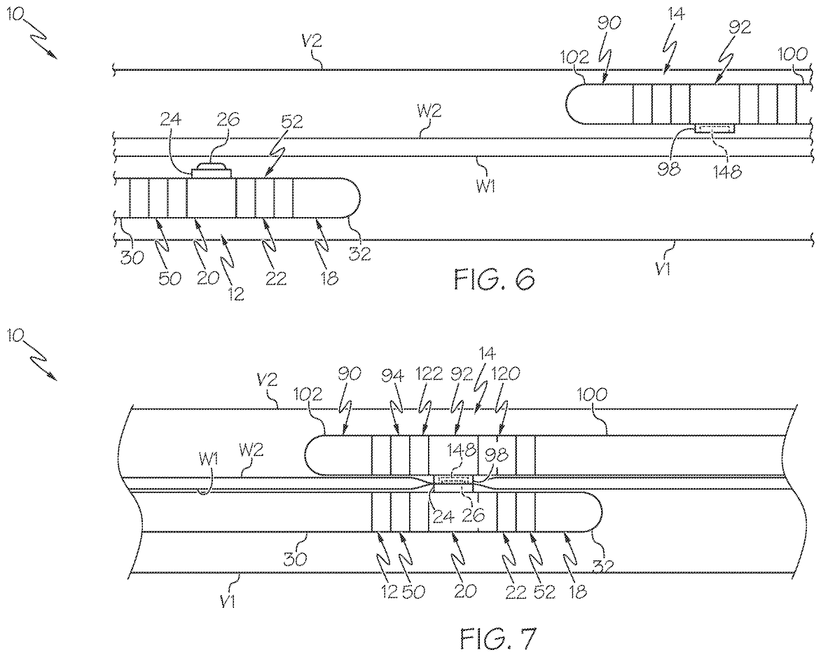

The operation of the catheter system 10 will now be described with reference to FIGS. 1-8. Referring initially to FIG. 6, the ablation catheter 12 may be inserted into the first vessel V1, and the coaptation catheter 14 may be inserted into the second vessel V2. Each of the ablation catheter 12 and the coaptation catheter 14 may be traversed through the respective first vessel V1 and the second vessel V2 to a treatment site. The treatment site may be an area of the first vessel V1 and the second vessel V2 where the fistula F is to be formed.

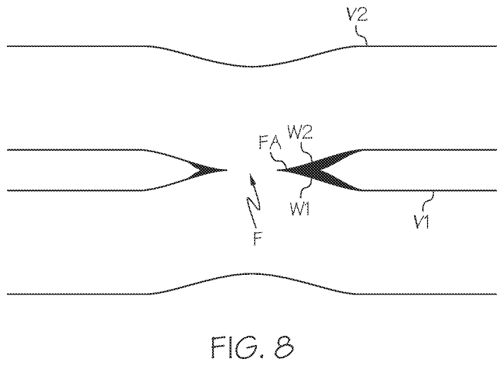

Referring to FIG. 7, when the ablation catheter 12 and the coaptation catheter 14 are positioned at the treatment site, the alignment elements 22 of the ablation catheter 12 are positioned adjacent the alignment elements 94 of the coaptation catheter 14 to coapt the ablation catheter 12 with the coaptation catheter 14. In the presently depicted embodiment, the proximal set of alignment elements 50 of the ablation catheter 12 coapts with the distal set of alignment elements 122 of the coaptation catheter 14, and the distal set of alignment elements 52 of the ablation catheter 12 coapts with the proximal set of alignment elements 120 of the coaptation catheter 14 to align the vessel-fusing element 98 of the coaptation catheter 14 with the vessel-fusing element 24 of the ablation catheter 12. When the vessel-fusing element 98 of the coaptation catheter 14 is aligned with the vessel-fusing element 24 (e.g., across from one another) of the ablation catheter 12, the fistula-forming element 26 of the ablation catheter 12 may be aligned with the opening 148 of the vessel-fusing element 98 of the coaptation catheter 14.

Once the coaptation catheter 14 is aligned with the ablation catheter 12, the generator 16 may be activated to energize the vessel-fusing element 24 of the ablation catheter 12 via the first conductor 66, and to energize the vessel-fusing element 98 of the coaptation catheter 14 via the conductor 96. When energized, each of the vessel-fusing elements 24, 98 may fuse the vessel wall W1 of the first vessel V1 to the vessel wall W2 of the second vessel V2. The vessel wall W1 of the first vessel V1 and the vessel wall W2 of the second vessel V2 may have a fused area that retains the vessel walls together. In embodiments, the vessel-fusing elements 24, 98 may fuse the vessel at any suitable temperature for fusing, such as between about 120° C. to about 170° C. The vessel-fusing elements 24, 98 may perform the fusing operation for a time sufficient to fuse the two vessel walls W1, W2 together, such between about 500 msec. to about 3000 msec. However, other temperatures and time periods are contemplated and possible.

After the fused area is formed between the vessel walls, the generator 16 may be activated to energize (such as with RF energy) the fistula-forming element 26 of the ablation catheter 12 via the second conductor 68. When the fistula-forming element 26 is energized, the fistula-forming element 26 ablates tissue, and via its natural bias, extend radially from the ablation catheter 12 through each of the vessel wall W1 of the first vessel V1 and the vessel wall W2 of the second vessel V2. The fistula-forming element 26 ablates tissue within a region bordered by the fused area to form the fistula F positioned within the fused area. Ablation temperatures may be higher than fusing temperatures such as greater than 170° C. That is, the fistula-forming element 26 may form the fistula F in the fused area so that the fused area surrounds the fistula. With the fused area surrounding the fistula, the first vessel V1 is fused to the second vessel V2 to prevent blood flow through the fistula from exiting both the first vessel V1 and the second vessel V2.

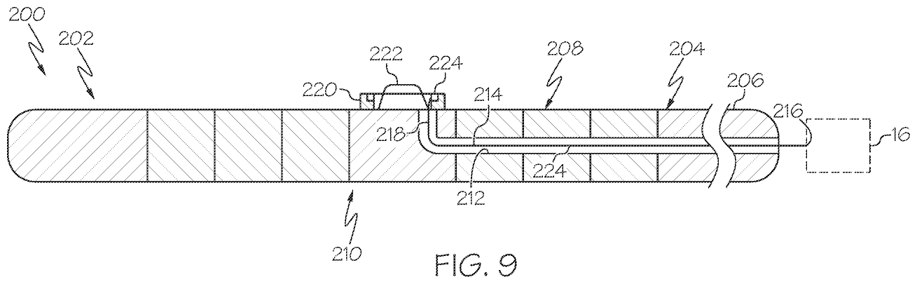

Referring now to FIG. 9, another catheter system 200 is depicted. The catheter system 200 includes substantially the same structure as the catheter system 10 described above. Accordingly, like elements will not be described again for brevity. The catheter system 200 differs from the catheter system described above in that an ablation catheter 202 of the catheter system 200 includes an ablation catheter body 204 having a proximal section 206, a proximal set of alignment elements 208, and an conductor housing 210 that define a common conductor lumen 212 that a common conductor 214, or common electrical pathway 214, is positioned in. The common conductor 214, or common electrical pathway 214, may include a proximal end 216 and an opposite distal end 218, the proximal end 216 being operatively coupled to the generator 16. The distal end 218 of the conductor 214 may be operatively coupled to both a conductive plate 224 of a vessel-fusing element 220 and a fistula-forming element 222 so that when the generator 16 energizes the conductor 214, both the vessel-fusing element 220 and fistula-forming element 222 are simultaneously energized to form both the fistula F and the fused area. The common electrical pathway 214 may transfers energy from the generator 16 to both the vessel-fusing element 220 and the fistula-forming element 222 so that a single actuation of the generator 16 causes the ablation catheter 202 to form both the fistula and the fused area at the same time.

In embodiments, the vessel-fusing element 220 and the fistula-forming element 222 may be operatively coupled together to be able to transfer energy between the vessel-fusing element 220 and the fistula-forming element 222. The operative coupling between the vessel-fusing element 220 and the fistula-forming element 222 allows the energized conductor 214 to transfer energy to one of the vessel-fusing element 220 and the fistula-forming element 222, and the one of the vessel-fusing element 220 and the fistula-forming element 222 transfers energy to the other of the vessel-fusing element 220 and the fistula-forming element 222. However, it is contemplated and possible that the conductor 214 is operatively coupled to both the vessel-fusing element 220 and the fistula-forming element 222 in other manners. For example, the conductor 214 may include a forked portion (not shown) at the distal end 218 of the conductor 214, with each of the forks connecting to a respective one of the vessel-fusing element 220 and the fistula-forming element 222.

In any of the embodiments described herein, a controller (not depicted), either as part of a handle of a catheter or the generator 16, may control energy delivery to the catheters. The controller may include one or more user input devices (e.g., toggles, buttons, switches, etc.) to input instructions for operation. In some embodiments, the controller may execute non-transitory computer-readable instructions, such as stored on a memory, to control operation of the system, based on user input. In one embodiment, the controller may execute instructions that cause the controller to operate the vessel-fusing element, as described herein for a first time period, at a first vessel-fusing temperature, and operate the fistula-forming element for a second time period at a second fistula-forming temperature. In some embodiments, the controller may execute instructions to cause the controller to operate both the vessel-fusing element and the fistula-forming element simultaneously. In both cases, the controller may controller, which may be communicatively coupled to the generator, may control generator settings to facilitate fusing and/or fistula formation (e.g., lowering or increasing energy delivery for the particular operation of vessel fusing or fistula formation). In some embodiments, the controller may receive a first user input via the one or more user input devices, and operate the vessel-fusing element in response to the first user input. The controller may subsequently receive a second user input via the one or more user input devices and operate the fistula-forming element in response to the second user input. In some embodiments, the controller may receive a single input from a user input device and may execute instructions to, either, sequentially or simultaneously operate the vessel-fusing element and the fistula-forming element.

Embodiments may be further described with reference to the following numbered clauses:
1. A catheter including a body, a vessel-fusing element coupled to the body and configured to fuse a pair of body vessels together, and a fistula-forming element positioned within and extending from the vessel-fusing element, the fistula-forming element configured to form a fistula between the pair of body vessels.
2. The catheter according to clause 1, further including a first electrical pathway coupled to the vessel-fusing element and a second electrical pathway coupled to the fistula-forming element.
3. The catheter according to either of clause 1 or 2, further including a common electrical pathway coupled to the vessel-fusing element and the fistula-forming element.
4. The catheter according to any of clauses 1-3, further including one or more alignment elements arranged adjacent to the vessel-fusing element.
5. The catheter according to any of clauses 1-4, further including a housing coupled to the body, wherein the vessel-fusing element extends from a surface of the housing.
6. The catheter according to any of clauses 1-5, wherein the vessel-fusing element defines a recess and the fistula-forming element is positioned within the recess.
7. The catheter according to any of clauses 1-6, wherein the vessel-fusing element includes a vessel-fusing housing defining a plate recess, and a conductive plate positioned within the plate recess of the vessel-fusing housing.
8. A catheter system including a first catheter and a second catheter. The first catheter includes a first body, a first vessel-fusing element coupled to the first body, and a fistula-forming element coupled to and extending from the first vessel-fusing element, the fistula-forming element configured to form a fistula between a pair of body vessels. The second catheter includes a second body, and a second vessel-fusing element coupled to the second body and configured to fuse the pair of body vessels together between the first vessel-fusing element and the second
9. The catheter system according to clause 8, wherein the second vessel-fusing element includes an opening formed therein configured to receive the fistula-forming element of the first catheter, and the first vessel-fusing element defines a recess and the fistula-forming element is positioned within the recess.
10. The catheter system according to either of clauses 8 or 9, further including a common electrical pathway coupled to the first vessel-fusing element and the fistula-forming element.
11. The catheter system according to any of clauses 8-10, further including a first electrical pathway coupled to the first vessel-fusing element and a second electrical pathway coupled to the fistula-forming element.
12. The catheter system according to any of clauses 8-11, wherein the first catheter further includes one or more first alignment elements arranged adjacent to the first vessel-fusing element, and the second catheter further includes one or more second alignment elements arranged adjacent to the second vessel-fusing element and configured to align with the one or more first alignment elements.
13. The catheter system according to any of clauses 8-12, wherein the first catheter further includes a first housing coupled to the first body, wherein the first vessel-fusing element extends from a first surface of the first housing, and the second catheter further includes a second housing coupled to the second body, wherein the second vessel-fusing element extends from a second surface of the second housing.
14. The catheter system according to any of clauses 8-13, wherein the first catheter further includes a first housing coupled to the first body, the first vessel-fusing element extends from a surface of the first housing, and the second catheter further includes a second housing coupled to the second body, the second vessel-fusing element extends from a surface of the second housing.
15. The catheter system according to clause 14, wherein the first vessel-fusing element includes a first vessel-fusing housing coupled to and extending from the first housing and a first conductive plate coupled to the first vessel-fusing housing, and the second vessel-fusing element includes a second vessel-fusing housing coupled to and extending from the second housing and a second conductive plate coupled to the second vessel-fusing housing.
16. The catheter system according to clause 15, wherein the first vessel-fusing housing defines a first plate recess and the first conductive plate is positioned within the first plate recess, and the second vessel-fusing housing defines a second plate recess and the second conductive plate is positioned within the second plate recess.
17. A method of operating a catheter system, the method including providing a first catheter, providing a second catheter, and actuating a generator electrically connected to a first electrical pathway to energize the first electrical pathway. The first catheter includes a first body, a first vessel-fusing element coupled to the first body, a fistula-forming element coupled to and extending from the first vessel-fusing element, the fistula-forming element configured to form a fistula between a pair of body vessels, and the first electrical pathway coupled to the first vessel-fusing element. The second catheter includes a second body, and a second vessel-fusing element coupled to the second body and configured to fuse the pair of body vessels together between the first vessel-fusing element and the second vessel-fusing element.
18. The method according to clause 17, further including actuating the generator electrically connected to a second electrical pathway to energize the second electrical pathway, wherein the second electrical pathway is coupled to the fistula-forming element of the first catheter.
19. The method according to either of clause 17 or 18, wherein the first electrical pathway is coupled to the fistula-forming element.
20. The method according to any of clauses 17-19, further including aligning a first alignment element of the first catheter with a second alignment element of the second catheter to locate the first catheter relative to the second catheter.
21. The fistula-forming element of any of clauses 1-20, wherein the fistula forming element is an electrode.
22. The vessel-fusing element of any of clauses 1-21, wherein the vessel-fusing element is a conductive plate.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter comprising:
a body;
a vessel-fusing element coupled to the body and configured to fuse a pair of body vessels together; and
a fistula-forming element positioned within and extending from the vessel-fusing element, the fistula-forming element configured to form a fistula between the pair of body vessels.

2. The catheter of claim 1, further comprising a first electrical pathway coupled to the vessel-fusing element and a second electrical pathway coupled to the fistula-forming element.

3. The catheter of claim 1, further comprising a common electrical pathway coupled to the vessel-fusing element and the fistula-forming element.

4. The catheter of claim 1, further comprising one or more alignment elements arranged adjacent to the vessel-fusing element.

5. The catheter of claim 1, further comprising a housing coupled to the body, wherein the vessel-fusing element extends from a surface of the housing.

6. The catheter of claim 5, wherein the vessel-fusing element defines a recess and the fistula-forming element is positioned within the recess.

7. The catheter of claim 5, wherein the vessel-fusing element comprises a vessel-fusing housing defining a plate recess, and a conductive plate positioned within the plate recess of the vessel-fusing housing.

8. A catheter system comprising:
a first catheter comprising:
a first body;
a first vessel-fusing element coupled to the first body;
a fistula-forming element coupled to and extending from the first vessel-fusing element, the fistula-forming element configured to form a fistula between a pair of body vessels;
a second catheter comprising:
a second body; and
a second vessel-fusing element coupled to the second body and configured to fuse the pair of body vessels together between the first vessel-fusing element and the second vessel-fusing element.

9. The catheter system of claim 8, wherein:
the second vessel-fusing element comprises an opening formed therein configured to receive the fistula-forming element of the first catheter, and
the first vessel-fusing element defines a recess and the fistula-forming element is positioned within the recess.

10. The catheter system of claim 8, further comprising a common electrical pathway coupled to the first vessel-fusing element and the fistula-forming element.

11. The catheter system of claim 8, further comprising a first electrical pathway coupled to the first vessel-fusing element and a second electrical pathway coupled to the fistula-forming element.

12. The catheter system of claim 8, wherein:
the first catheter further comprises one or more first alignment elements arranged adjacent to the first vessel-fusing element, and
the second catheter further comprises one or more second alignment elements arranged adjacent to the second vessel-fusing element and configured to align with the one or more first alignment elements.

13. The catheter system of claim 8, wherein:
the first catheter further comprises a first housing coupled to the first body, wherein the first vessel-fusing element extends from a first surface of the first housing, and
the second catheter further comprises a second housing coupled to the second body, wherein the second vessel-fusing element extends from a second surface of the second housing.

14. The catheter system of claim 8, wherein:
the first catheter further comprises a first housing coupled to the first body, the first vessel-fusing element extends from a surface of the first housing, and
the second catheter further comprises a second housing coupled to the second body, the second vessel-fusing element extends from a surface of the second housing.

15. The catheter system of claim 14, wherein:
the first vessel-fusing element comprises a first vessel-fusing housing coupled to and extending from the first housing and a first conductive plate coupled to the first vessel-fusing housing, and
the second vessel-fusing element comprises a second vessel-fusing housing coupled to and extending from the second housing and a second conductive plate coupled to the second vessel-fusing housing.

16. The catheter system of claim 15, wherein:
the first vessel-fusing housing defines a first plate recess and the first conductive plate is positioned within the first plate recess, and
the second vessel-fusing housing defines a second plate recess and the second conductive plate is positioned within the second plate recess.

17. A method of operating a catheter system, the method comprising:
providing a first catheter comprising:
a first body;
a first vessel-fusing element coupled to the first body;
a fistula-forming element coupled to and extending from the first vessel-fusing element, the fistula-forming element configured to form a fistula between a pair of body vessels;
a first electrical pathway coupled to the first vessel-fusing element;
providing a second catheter comprising:
a second body; and
a second vessel-fusing element coupled to the second body and configured to fuse the pair of body vessels together between the first vessel-fusing element and the second vessel-fusing element; and
actuating a generator electrically connected to the first electrical pathway to energize the first electrical pathway.

18. The method of claim 17, further comprising:
actuating the generator electrically connected to a second electrical pathway to energize the second electrical pathway,
wherein the second electrical pathway is coupled to the fistula-forming element of the first catheter.

19. The method of claim 17, wherein the first electrical pathway is coupled to the fistula-forming element.

20. The method of claim 17, further comprising:
aligning a first alignment element of the first catheter with a second alignment element of the second catheter to locate the first catheter relative to the second catheter.
